(19) United States
(12) Patent Application Publication
(10) Pub. No.: US 2026/0207886 A1
(43) Pub. Date: Jul. 23, 2026

(54) NOVEL PERIPHERALLY-INSERTED CENTRAL CATHETER

(71) Applicant: Wonseon SHEEN, Goyang-si, Gyeonggi-do (KR)

(72) Inventor: Wonseon SHEEN, Goyang-si, Gyeonggi-do (KR)

(21) Appl. No.: 19/138,565

(22) PCT Filed: Nov. 17, 2023
(86) PCT No.: PCT/KR2023/018562

§ 371 Date: Jun. 12, 2025

(30) Foreign Application Priority Data
Dec. 12, 2022 (KR) 10-2022-0172450
Nov. 2, 2023 (KR) 10-2023-0150081

(51) Int. Cl. A61M 25/00; A61M 25/06; A61M 25/09

(52) U.S. Cl. CPC A61M 25/0026; A61M 25/007; A61M 25/09; A61M 2025/0687

(57) ABSTRACT

Provided is a peripherally inserted central catheter (PICC) that, unlike conventional insertion methods, can be more easily inserted into a patient's body without damaging skin tissue or blood vessels during the procedure by applying a catheter having a blunt, tipless distal end structure and using only a dilator without a peel-away sheath.

### TECHNICAL FIELD

The present invention relates to a novel peripherally inserted central catheter (PICC).

### BACKGROUND ART

The insertion of a peripherally inserted central catheter (PICC) is a technique designed to allow blood collection, transfusion, fluid administration, and drug delivery to be performed in a less painful, simpler, and more effective manner. The procedure involves puncturing a peripheral vein, temporarily inserting a micro-guide wire into the vessel, and then introducing a catheter thinner than standard central venous catheters along the micro-guide wire, to advance it into the superior vena cava. The tip of the catheter is positioned at or near the junction of the superior vena cava and the heart. PICC placement is typically performed in patients who require long-term catheter use or in those whose veins are difficult to access due to extended illness. In these cases, the brachial vein in the upper arm is identified using ultrasound, and the catheter is inserted usually under local anesthesia, through either the right or left arm depending on vascular conditions. This procedure allows for relatively easy placement and low complication rates, and can be performed at the bedside without requiring transport to an interventional suite or operating room equipped with angiographic or fluoroscopic devices. It can be used for a long term of 2-3 months and is easily replaceable if extended use is needed. Because the catheter tip resides in the central vein, it can be used to infuse corrosive solutions with low pH or high osmotic pressure. It is applicable for both inpatient and outpatient care. Studies have shown that PICC usage improves patient satisfaction, and reduces hospital stay duration and thus costs. Therefore, PICCs are considered suitable for patients who need prolonged drug administration or infusion of corrosive agents, and they are most commonly used in long-term antibiotic therapy, total parenteral nutrition, blood transfusion, and chemotherapy. In this regard, Korean Patent Publication No. 2022-0086926 discloses a peripherally inserted central catheter (PICC) having both a lumen for injecting saline solution and drugs, and a lumen for collecting blood.

### DETAILED DESCRIPTION OF THE INVENTION

### Technical Problem

However, current PICCs including those of the above prior art are designed to be inserted and placed using a dilator/peel-away sheath assembly. The peel-away sheath used during the procedure is removed after catheter placement. Because the diameter of the catheter is smaller than that of the peel-away sheath, the removement of the sheath leaves a void at the insertion site, between the catheter and the catheter-surrounding area from the blood vessel wall to the skin surface. As a result, bleeding frequently occurs at the catheter insertion site after placement.

If an attempt is made to insert the catheter without using the dilator/peel-away sheath assembly to reduce such bleeding, a gap may form due to the difference in diameter between the micro-guide wire and the distal end of the PICC, which may damage skin tissue or the vessel wall during insertion. This may hinder smooth insertion, and in some cases, insertion may be impossible. The present invention is intended to solve the above-mentioned and other problems. Unlike the conventional method using a peel-away sheath, the present invention employs a catheter with a blunt distal end which is designed to be inserted using only a dilator. Accordingly, the objective is to provide a novel peripherally inserted central catheter (PICC) that can be more easily inserted into the patient's body without damaging skin tissue or blood vessels during the procedure. However, these problems are only exemplary and the scope of the present invention is not limited thereto.

### Technical Solution

According to one aspect of the present invention, there is provided a catheter kit for peripheral vascular insertion comprising: a catheter body portion having a cylindrical structure configured to be inserted through a human blood vessel and having a lumen extending entirely therethrough;
a perfusion side hole formed on one side of the catheter body portion and configured to discharge an injected fluid, which is introduced from the catheter body portion into the human body;
a catheter distal end portion extending from the catheter body portion and having a tapering structure that gradually narrows toward its terminal end;
a peripherally inserted central catheter comprising an end hole for insertion of a micro-guided wire which is formed at the center of the catheter distal end portion; and
a dilator configured to dilate the vessel lumen by being inserted into the blood vessel.

According to another aspect of the present invention, there is provided a catheter kit for peripheral vascular insertion comprising: a catheter body portion having a cylindrical structure configured to be inserted through a human blood vessel and having a lumen extending entirely therethrough; a peripherally inserted central catheter comprising a perfusion side hole formed on a side of the catheter body portion for discharging an injected fluid, which has been introduced from inside the catheter body portion into the human body; and
a stiffener comprising a body insertable into the interior of the catheter body portion, a tapered distal end portion formed at the terminal end of the body, and an end hole formed at the center of the body for insertion of a micro-guide wire.

### Effect of the Invention

The novel peripherally inserted central catheter (PICC) of the present invention, constructed as described above, provides the advantage of enabling rapid catheter insertion while minimizing tissue and vascular wall damage—and thus reducing the risk of bleeding—due to its unique structure featuring a blunt distal end and the use of only a dilator unlike conventional catheters. However, the scope of the present invention is not limited by these effects.

### BRIEF DESCRIPTION OF THE DRAWINGS

FIG. 1 is a perspective view schematically showing the structure of the novel PICC of the present invention.

FIG. 2 is a cross-sectional view of the novel PICC composed of a single lumen.

FIG. 3 is a cross-sectional view of the novel PICC composed of a double lumen.

FIG. 4 is a longitudinal sectional view of the novel PICC composed of a double lumen.

FIG. 5 is a longitudinal sectional view of the novel PICC composed of a triple lumen with a T-shaped internal structure.

FIG. 6 is a cross-sectional view of the novel PICC composed of a triple lumen.

FIG. 7 is a longitudinal sectional view of the novel PICC composed of a triple lumen with a Y-shaped internal structure.

FIG. 8 is a cross-sectional view showing the catheter body portion of the novel PICC with a stiffener inserted.

FIG. 9 is a cross-sectional view showing a stiffener inserted into the catheter body portion of a double-lumen or triple-lumen PICC of the present invention.

FIG. 10 is a sectional view illustrating a modified example of the internal structure of the novel PICC composed of a double lumen.

FIG. 11 is a photograph showing a combined unit of a dilator and peel-away sheath used during PICC insertion.

FIG. 12 is a cross-sectional view of the novel PICC composed of a double lumen and an internal space (204) filled with a filler material.

FIG. 13 is a sectional view illustrating another modified internal structure of the novel PICC composed of a double lumen.

### BEST MODE FOR CARRYING OUT THE INVENTION

### Detailed Description of the Invention

According to one aspect of the present invention, there is provided a catheter kit for peripheral vascular insertion comprising: a catheter body portion having a cylindrical structure configured to be inserted through a human blood vessel and having a lumen extending entirely therethrough;
a perfusion side hole formed on one side of the catheter body portion and configured to discharge an injected fluid, which is introduced from the catheter body portion into the human body;
a catheter distal end portion extending from the catheter body portion and having a tapering structure that gradually narrows toward its terminal end;
a peripherally inserted central catheter comprising an end hole for insertion of a micro-guided wire which is formed at the center of the catheter distal end portion; and
a dilator configured to dilate the vessel lumen by being inserted into the blood vessel.

In the kit, a peel-away sheath may be omitted. The dilator may have a diameter equal to or smaller than that of the peripherally inserted central catheter, and more specifically, may have a diameter reduced by up to 10% compared to the diameter of the PICC.

In the kit, the diameter of the end hole may be greater than the diameter of the micro-guide wire to be inserted, and more specifically, may be increased by up to 10% relative to the diameter of the micro-guide wire. The interior of the catheter body portion may be divided by one or more internal partitions, and depending on the shape of the partitions, the catheter body portion may be configured as a single lumen, double lumen, triple lumen, or multiple lumens.

In the kit, the double lumen may include a main lumen that extends through the entire catheter body portion, and a sub-lumen which is separated from the main lumen by the internal partition and includes a blocking partition located in front of the side hole. The triple lumen may include a single main lumen extending through the entire catheter body portion, and a plurality of sub-lumens, which are separated from the main lumen by two or more internal partitions and include blocking partitions in front of the side hole.

In the kit, the step difference between the end hole and the internal partitions may be minimized by filling the lower spaces in front of the catheter distal end portion and the blocking partitions with a material that is the same or similar to that of the catheter body portion. Alternatively, the lower portion of the tubular body in front of the catheter distal end portion and blocking partition may be integrally structured in a thick form. The material may be a thermoplastic polymer, thermosetting polymer, or biodegradable polymer. The thermoplastic polymer may be selected from the group consisting of silicone, polyurethane (PU), polytetrafluoroethylene (PTFE, Teflon), polyvinyl chloride (PVC), polystyrene, nylon, polyethylene terephthalate (PET), polyacrylate, polypropylene (PP), polyethylene (PE), polycarbonate (PC), polyetheretherketone (PEEK), and polysulfone (PS). The thermosetting polymer may be selected from the group consisting of phenol resin and epoxy resin. The biodegradable polymer may be selected from the group consisting of polyglycolide (PGA), poly-L-lactide (PLLA), polycaprolactone (PCL), PLGA, PLCL, polydioxanone (PDO), polytrimethylene carbonate (PTMC), polyanhydride, polyorthoester, polyphosphaphazene, and their copolymers.

In the kit, the double lumen may comprise a main lumen that extends through the entire catheter body portion; and a shorter sub-lumen which is separated from the main lumen by an internal partition and has a blocking partition at its terminal end.

The double lumen may allow for insertion of a micro-guide wire when external force is applied in the vertical or horizontal direction, as a space may be formed at the center of the internal partition. The wall thicknesses of the catheter body portion and the catheter distal end portion may be identical. The injectable material introduced into the interior of the catheter body portion may be blood, an infusion solution, or a drug.

According to another aspect of the present invention, there is provided a catheter kit for peripheral vascular insertion comprising: a catheter body portion having a cylindrical structure configured to be inserted through a human blood vessel and having a lumen extending entirely therethrough; a peripherally inserted central catheter comprising a perfusion side hole formed on a side of the catheter body portion for discharging an injected fluid, which has been introduced from inside the catheter body portion into the human body; and
a stiffener comprising a body insertable into the interior of the catheter body portion, a tapered distal end portion formed at the terminal end of the body, and an end hole formed at the center of the body for insertion of a micro-guide wire.

In the kit, the diameter of the end hole may be increased by about 20% to 30% relative to the diameter of the micro-guide wire to be inserted, and preferably increased by 10% or less. The outer diameter of the stiffener and the inner diameter of the catheter body portion may be the same; alternatively, the stiffener may have an outer diameter increased by 20% to 30% compared to the inner diameter of the catheter body portion, and preferably decreased by 10% or less.

Hereinafter, preferred embodiments of the present invention will be described in detail with reference to the accompanying drawings.

The embodiments of the present invention are provided to more fully explain the invention to those skilled in the art and may be modified in various other forms. The scope of the present invention is not limited to the embodiments described herein. Rather, the embodiments are provided to more completely describe the disclosure and to fully convey the concept of the invention to those skilled in the art. Also, the thickness and size of each layer in the drawings may be exaggerated for clarity and ease of explanation.

Throughout the present specification, when one element such as a membrane, area, or substrate is referred to as being “on”, “connected to”, “stacked on”, or “coupled to” another element, it may be interpreted as being in direct contact with—for example, being “on”, “connected to”, “stacked on”, or “coupled to”—the other element, or with one or more intervening elements interposed therebetween. In contrast, when one element is referred to as being “directly on”, “directly connected to”, or “directly coupled to” another element, it should be understood that no intervening elements exist in between. Particular reference numbers refer to particular elements. As used herein, the term “and/or” includes any one or more possible combinations of the listed elements.

Although the terms “the first,” “the second,” and the like may be used to describe various elements, components, regions, layers, and/or parts, these elements, components, regions, layers, and/or parts should not be limited by these terms. These terms are used only to distinguish one element, component, region, layer, or part from another. Thus, a “the first” element, component, region, layer, or part may be referred to as a “the second” element, component, region, layer, or part without departing from the teachings of the present invention.

Additionally, relative terms such as “above,” “on,” “below,” and “under” may be used to describe the relationship between components as illustrated in the drawings. These relative terms are intended to encompass different orientations in addition to the orientation depicted in the drawings. For example, if a component is flipped over in the drawing, elements described as “above” another element may become “below” it. Therefore, the term “above” may encompass both “above” and “below” depending on the orientation. If the component directs at different direction, for example, if it is rotated by 90 degrees, the relative descriptions herein are to be interpreted accordingly.

The terminology used herein is for the purpose of describing particular embodiments only and is not intended to be limiting. As used herein, the singular forms “a,” “an,” and “the” include plural references unless the context clearly dictates otherwise. Further, the terms “comprise” and/or “comprising,” as used herein, specify the presence of stated features, numbers, steps, operations, elements, and/or components but do not preclude the presence or addition of one or more other features, numbers, steps, operations, elements, components, and/or groups thereof.

Hereinafter, embodiments of the present invention will be described with reference to the drawings, which schematically illustrate ideal embodiments of the invention. In the drawings, for example, due to manufacturing techniques and/or tolerances, variations in the illustrated shapes may be expected. Therefore, the embodiments of the invention should not be construed as limited to the specific illustrated shapes but should be understood to include variations caused by manufacturing deviations.

The conventional procedure for inserting a novel peripherally inserted central catheter (PICC) will be described in detail. Under ultrasound guidance, the vein in the antecubital area, such as the basilic vein or brachial vein located on the upper side of the forearm, is punctured through its anterior wall using a micropuncture needle with a beveled tip, typically with a thickness of 21G or 22G. Once the needle tip is located within the lumen of the punctured vessel, blood flow is confirmed through the hub at the opposite end of the micropuncture needle. A 0.018″ micro-guidewire is advanced through the hub until its distal end is positioned deep within the lumen of the target vessel. Then, only the micropuncture needle is removed from the body. Subsequently, a dilator having a tapered distal end portion is inserted into a peel-away sheath prepared to match the outer diameter of the intended PICC, forming a peel-away introducer assembly. The assembly is then inserted along the micro-guidewire, expanding the access path through the skin, tissue, and vessel wall, and the peel-away sheath is advanced into the lumen of the target vein.

If fluoroscopic guidance is available, the micro-guidewire may be positioned into the superior vena cava (SVC) or at the SVC-right atrium (RA) junction. The insertion depth from the skin entry site to the wire tip is measured using external markings on the wire, and a PICC of corresponding length is prepared. The PICC tip is beveled and inserted so that the hub contacts the skin and the PICC tip aligns with the distal end of the micro-guidewire. If fluoroscopy is unavailable, distance between the insertion site and the armpit, and that between the armpit and the right atrium may be estimated from the outside or determined empirically without such measurement by the practitioner, and the tip may be beveled accordingly. The dilator is removed from the introducer assembly, leaving the guidewire and peel-away sheath in place, and the prepared PICC is advanced over the wire through the sheath into the vessel. After full advancement, the peel-away sheath is split and removed, and the PICC is inserted until the hub contacts or nearly contacts the skin. The guidewire is then withdrawn. If the PICC tip is inserted too deeply or not far enough, the tip may be trimmed and reinserted, or a new PICC may be prepared and inserted at the correct length.

Thereafter, the number of syringe or infusion line connectors corresponding to the number of lumens in the inserted PICC can be used to verify the proper function of each lumen, including smooth drainage and infusion of blood or fluids. If necessary, contrast media may be injected under fluoroscopic guidance to confirm proper function based on the position of the PICC tip in relation to surrounding vascular structures. Subsequently, Thereafter, the PICC is secured to the skin by either suturing it through the hole of the wing coupled to the hub or by fitting the hub into the hole of a skin attachment device and attaching it to the skin, or alternatively, by first attaching the skin attachment device to the skin in alignment with the desired position and then inserting the hub so that the skin attachment device aligns with the hole, thereby securing the hub to the skin and completing the insertion of the PICC. However, procedures using conventional PICCs as described above have the following problems. Upon completion of these steps, insertion of the PICC is finalized. However, the above procedures using the conventional PICC still involves the following problems.

First, the inner diameter of the peel-away sheath (106) is typically manufactured to closely match or slightly exceed the outer diameter of the dilator (105), often by 0.5 to 1.0 Fr (0.167 to 0.333 mm). This mismatch creates a discontinuity at the junction of the sheath and dilator, which can damage tissue, skin, or vessel walls during insertion, leading to bleeding. Furthermore, because the access path is expanded to match the outer diameter of the sheath (106) rather than the dilator (105), after leaving a thinner PICC in place where the dilator (105) is removed and then removing the sheath (106), a small gap due to the outer diameter difference between the sheath (106) and the PICC catheter remains, potentially causing bleeding around the insertion site. In cases of significant bleeding, manual compression for hemostasis may enable bleeding control within one minute in more than half of cases; however, in some cases, compression may need to be applied for one to ten minutes or longer. There are even instances where manual compression must be continued while the patient is being transported to the hospital room. To address such bleeding issues, a reverse tapered PICC is sometimes used; however, studies have reported that such designs may increase the risk of thrombosis. In light of these problems, the present inventors have developed a novel peripherally inserted central catheter (100) that overcomes the limitations of conventional PICC procedures, particularly tissue and vascular damage as well as bleeding complications.

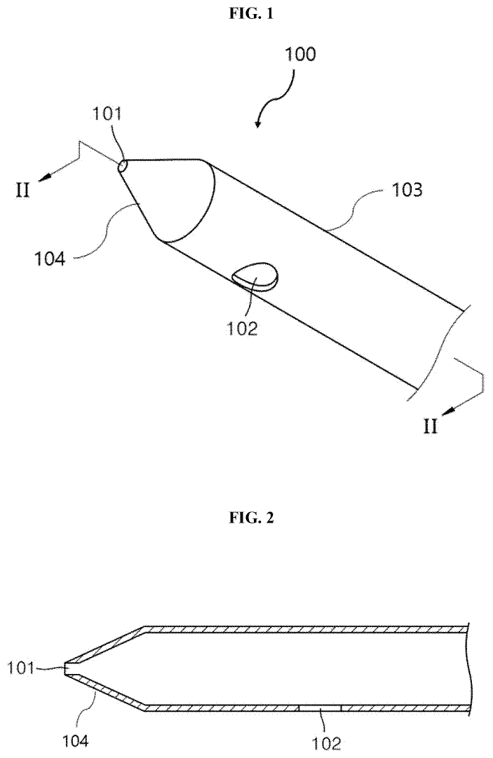

FIG. 1 schematically illustrates the structure of the novel PICC (100) of the present invention, which consists of a catheter body portion (103) having an internal lumen and a tapered catheter distal end portion (104). A perfusion side hole (102) is formed on one side of the catheter body portion (103) to allow fluid delivery after insertion. The catheter distal end portion (104) has a conical taper extending from the end of the catheter body, and at its center, an end hole (101) is formed to allow the insertion of a micro-guidewire through the catheter body portion (103). The end hole (101) may have a diameter equal to or slightly greater than that of the micro-guidewire (typically 0.36 to 0.51 mm) and may be manufactured larger or smaller depending on the micro-guidewire. More specifically, the diameter may be increased by up to 10% over the micro-guidewire diameter, but is not limited to this.

Additionally, although FIG. 1 illustrates a configuration in which the perfusion-side first side hole (102) is positioned near the catheter distal end portion (104) of the catheter, the side hole may be formed on any outer side of the catheter body portion (103) depending on the manufacturer's design intent. One or more side holes may be provided depending on the intended use. Typically, the diameter of the side hole is between 2 mm and 4 mm; however, it may also be manufactured to be larger or smaller to enhance the effectiveness of the procedure.

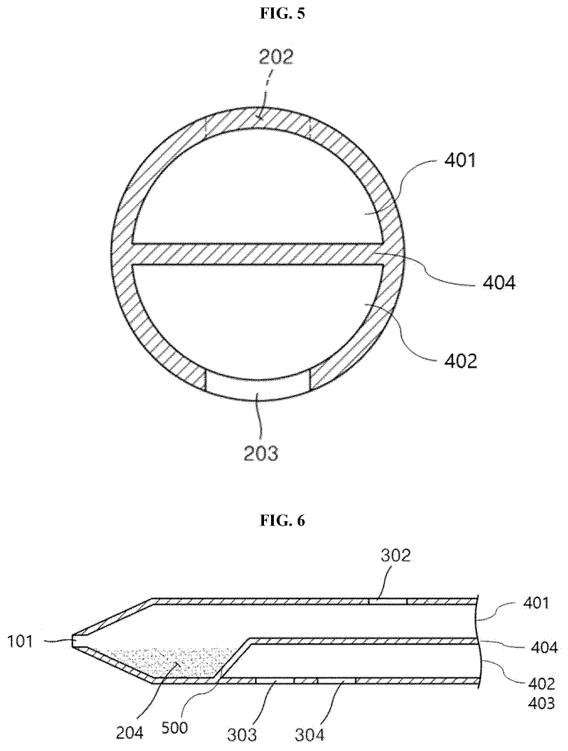

As described above, the present invention is characterized in that, during catheter insertion, a dilator (105) having an outer diameter equal to or slightly smaller than the outer diameter of the PICC is used, rather than the conventional one which matches the outer diameter of the peel-away sheath (106). Accordingly, the vessel can be dilated using only the dilator (105) without employing the peel-away sheath (106), allowing insertion of the novel peripherally inserted central catheter (100) of the present invention. In other words, instead of inserting a dilator (105) in combination with a peel-away sheath (106) as in conventional introducer assemblies (110), only the dilator (105) is inserted after micro-guidewire placement to dilate the vessel, and the novel PICC (100) is then advanced through the vessel. Therefore, the present invention prevents bleeding that may be caused by the gap between the peel-away sheath (106) and the PICC due to the outer diameter difference, which arises when using the conventional introducer assembly (110). The outer diameter of the dilator (105) may be equal to or reduced by 20% to 30% relative to the outer diameter of the novel PICC (100); preferably, the reduction may be within 10%, and more specifically within 10% to 0%, 7% to 0%, 5% to 0%, 3% to 0%, or 1% to 0%. However, when the peel-away sheath (106) is not used, cutting the tip of the PICC may create a raised spot of a sharp edge, which can catch at the skin entry site or vascular entry point during insertion, potentially causing insertion failure or requiring multiple attempts. For example, although the commercial Teleflex's Arrow PICCs are made from flexible materials with a tapered structure, the degree of tapering is insufficient. Also, due to the performance-driven structural design, the diameter of the end hole is relatively large. Therefore, when the catheter is inserted without the peel-away sheath (106), the gap between the micro-guidewire and the end hole may result in the catheter catching on tissue or the vessel wall, making insertion difficult and requiring several attempts. Furthermore, while conventional PICCs composed of a single lumen can generally center the micro-guidewire within the lumen depending on its diameter, in dual-lumen (see FIG. 4) or triple-lumen (see FIG. 5) PICCs, the inherent structure may prevent the micro-guidewire from being centered within the catheter, further exacerbating the issue of catching on the vessel wall. Accordingly, to address these problems, the present inventors have developed the novel peripherally inserted central catheter (100) of the present invention, comprising a catheter distal end portion (104) that gradually tapers toward the tip, enabling improved insertion performance.

FIG. 2 illustrates a cross-sectional view of the novel peripherally inserted central catheter (100) of the present invention configured as a single lumen. As described above, unlike conventional structures, the catheter adopts a catheter distal end portion (104) that tapers steeply and narrows from the end of the catheter body portion (103), enabling the micro-guidewire to be centered within the lumen during insertion. Because the end of the lumen is not cut, the raised spot is not formed at the gap or protrusion of the end hole (101), thereby preventing the catheter from catching at the skin or vessel wall entry points, which could otherwise lead to damage. Meanwhile, the tapered structure of the catheter distal end portion (104) adopted in the present invention must not be excessively rigid. If it is too stiff, prolonged indwelling within a blood vessel may cause injury to the vessel wall due to movement caused by cardiac pulsations. Therefore, appropriate flexibility and hardness are required to ensure safe vascular insertion. Additionally, as shown in FIG. 2, the thickness of the sidewalls forming the catheter body portion (103) and the catheter distal end portion (104) may remain uniform up to the end hole (101), rather than gradually decreasing from the body to the catheter distal end portion, in other words, from the proximal part to the distal part.

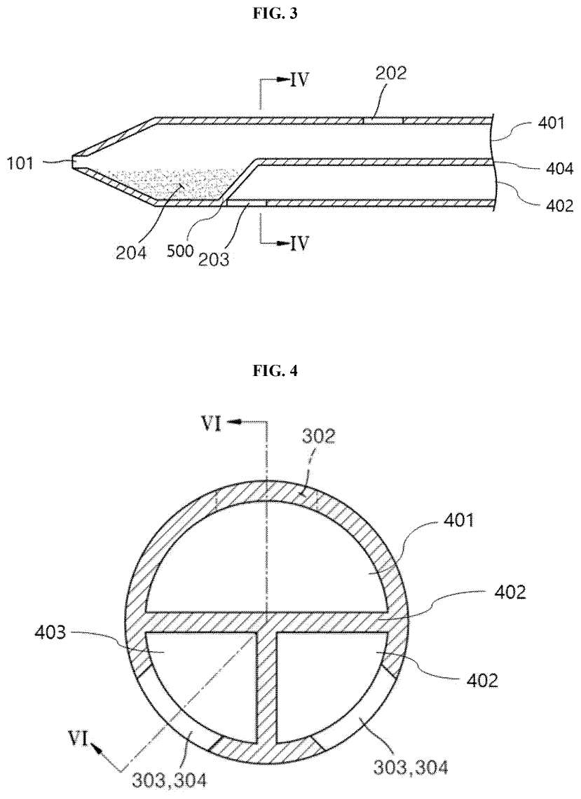

FIG. 3 illustrates a cross-sectional view of the novel peripherally inserted central catheter (100) of the present invention configured as a double lumen. As shown, the external shape of the catheter body portion (103) and the catheter distal end portion (104) is the same as that of the single-lumen configuration; however, the internal structure of the lumen differs in that it is composed of a double lumen. By adopting the double lumen configuration, the second perfusion side hole (202) and the third perfusion side hole (203), corresponding to each lumen, are formed on one side of the catheter body portion (103), through which blood, infusion solution, or drugs are delivered into the patient's body. The interior of the catheter body portion (103) configured as a double lumen includes the first lumen (401), which may be referred to as the main lumen, and the second lumen (402), which may be referred to as the sub-lumen. As illustrated, the first lumen (401) is formed to extend from the catheter body portion (103) through to the end hole (101); however, the second lumen (402) is separated from the main lumen by an internal partition (404), and a blocking partition (500) is formed in front of the third side hole (203), preventing it from communicating with the end hole (101). The double lumen structure may be configured such that the step between the end hole (101) and the internal partition (404) is minimized by filling the lower space (internal space, 204) located in front of the catheter distal end portion (104) and the blocking partition (500) with a material that is identical or similar to that of the catheter body. Alternatively, the lower portion of the tubular body located in front of the catheter distal end portion (104) and the blocking partition (500) may be integrally structured in a thick form. In this regard, FIG. 12 illustrates a cross-sectional view of the novel peripherally inserted central catheter of the present invention configured as a double lumen, with its internal space (204) filled with a filler material. The filler is preferably made from a material having the same or higher strength and hardness compared to that used in conventional PICCs; for example, a thermoplastic polymer such as medium- or high-hardness plastic, polyurethane, or silicone may be used.

Because there is no internal partition (404) at the end of the catheter body portion (103), the structure may be lightweight and lack sufficient support, making it susceptible to bending during insertion. By including a filler in the internal space (204), the support structure is reinforced, preventing bending and facilitating insertion into the blood vessel.

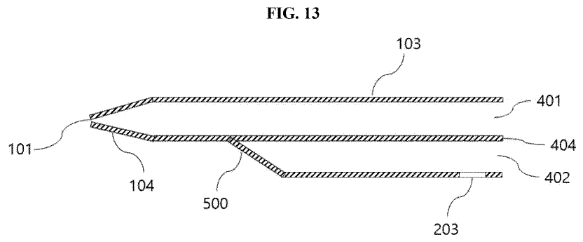

FIG. 13 illustrates a cross-sectional view showing a modified example of the internal structure of the novel peripherally inserted central catheter (100) of the present invention configured as a double lumen. As shown, in FIG. 13, the sub-lumen, or the second lumen (402), is formed to be shorter than the main lumen, or the first lumen (401); that is, the portion corresponding to the internal space (204) in the conventional double-lumen structure (FIG. 3) is removed, and a blocking partition (500) is formed at the end. Due to this structure, compared to the general double-lumen configuration shown in FIG. 3, the catheter distal end portion (104) of the first lumen (401) has a smaller and thinner diameter, allowing it to more easily pass through the skin entry, tissue, and vascular entry sites that were dilated by the dilator. As a result, the insertion success rate increases, and the insertion time is reduced. The side hole (203) formed in the second lumen (402), which is the sub-lumen, may be manufactured on one side of the second lumen (402) depending on the manufacturer's intent; more specifically, it may be formed close to or spaced apart from the blocking partition (500). With reference to FIG. 3, it can be seen that one side hole is provided for each lumen. Generally, in double-lumen or triple-lumen catheters, where the number of catheter lumens is two or three, respectively, it is possible to manufacture catheters having one or more side holes for each lumen.

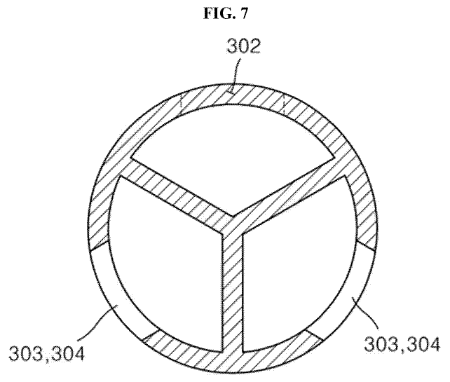

FIG. 4 illustrates a longitudinal sectional view of the novel peripherally inserted central catheter (100) of the present invention configured as a double lumen. After the novel PICC (100) is inserted into a patient, selective infusion of substances such as blood, infusion solutions, or drugs is possible through the double lumen. Of course, the internal structure of the lumen may also be manufactured in a triple-lumen configuration, such as the T-shaped structure shown in FIG. 5 or the Y-shaped structure shown in FIG. 7.

FIG. 6 is a cross-sectional view showing a triple-lumen configuration of the novel peripherally inserted central catheter (100) of the present invention. As illustrated, the catheter adopts a triple-lumen structure, and each of the lumens is provided with a corresponding perfusion side hole: the fifth side hole (302), sixth side hole (303), and seventh side hole (304).

The interior of the catheter body portion (103) configured as a triple lumen includes the first lumen (401), referred to as the main lumen, and the second and third lumens (402 and 403), referred to as sub-lumens. Like the double-lumen configuration, blocking partitions (500) are formed near the end of the catheter body portion (103) in the second and third lumens (402 and 403), respectively, to prevent communication with the end hole (101). Although only double-lumen and triple-lumen configurations are described in the drawings for ease of understanding the features of the invention, the triple-lumen and even higher multi-lumen configurations may also be manufactured with the single main lumen and a plurality of additional sub-lumens.

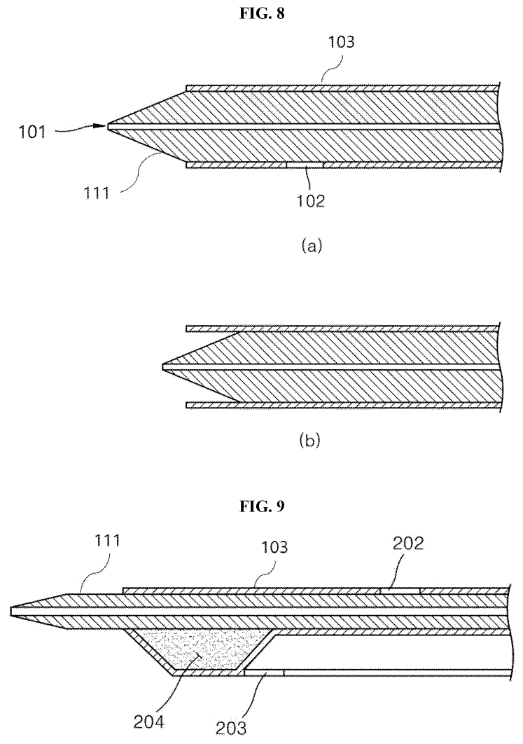

FIG. 8 is a cross-sectional view showing the catheter body portion (103) of the novel peripherally inserted central catheter (100) of the present invention with a stiffener (111) inserted therein. Unlike the previous examples, FIG. 8 illustrates a configuration in which the stiffener (111), manufactured as an integrated structure comprising both the distal end and body portions, is inserted directly into the catheter body portion (103) without the use of a dilator (105). The diameter of the end hole (101) formed within the stiffener (111) may be approximately equal to the diameter of the micro-guidewire, or may be manufactured to be 10 to 20% larger than the diameter of the micro-guidewire. Accordingly, there is either no gap or only a very slight gap between the micro-guidewire and the end hole (101), which does not cause tissue or vessel wall damage. Therefore, the use of the stiffener (111) enables the same ease of insertion as described earlier. The stiffener (111) is temporarily inserted into the catheter body portion (103) during the insertion procedure (a), and after completion of the procedure, can be quickly removed from the catheter body portion (103) (b). Considering that the stiffener (111) is temporarily inserted and removed, it is preferably made of a material that has flexibility so that the tissue and vessel wall are not damaged upon the insertion, and that has higher strength and hardness than conventional PICCs to allow smooth advancement without bending due to the vessel. For example, it may be made of a thermoplastic polymer such as medium- or high-hardness plastic, polyurethane, or silicone.

FIG. 9 is a cross-sectional view showing the catheter body portion (103) of the novel peripherally inserted central catheter (100) of the present invention, configured as a double or triple lumen, with a stiffener (111) inserted. As illustrated, the configuration combines the advantage of the stiffener (111), which can be temporarily inserted and removed during the insertion procedure, with the advantage of a multi-lumen structure capable of delivering blood, infusion fluids, and pharmaceutical agents through multiple lumens. The internal space (204) shown in the figure may remain empty or may be filled with a filler material. The filler is preferably made from a material with strength and hardness equal to or greater than that of conventional PICCs, such as medium- or high-hardness plastic, polyurethane, or silicone.

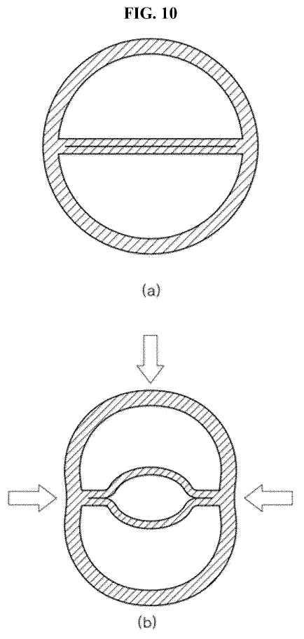

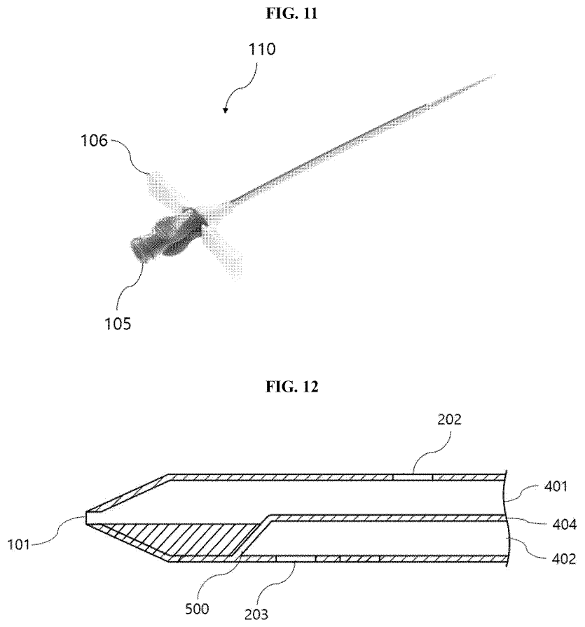

FIG. 10 illustrates a modified example of the internal structure of the novel peripherally inserted central catheter (100) of the present invention configured as a double lumen. The figure shows the internal structure of the double-lumen configuration, in which the central region remains normally closed (a), but when insertion of a micro-guidewire is required, pressure may be applied to the left and right or upper and lower parts of the catheter to cause the deformation of the central partition, thereby creating a space (b). This space enables easier insertion of the micro-guidewire. To accommodate this deformation, the catheter body portion (103) may be manufactured from a flexible material that allows the central internal structure to deform and form a space when manual pressure is applied. Although FIG. 10 illustrates the space formed in the center somewhat exaggerated for the sake of understanding the structural features of the invention, in actual use, the size of the space may correspond to the diameter of the micro-guidewire or may be slightly larger, for example, within a range of 20 to 30% greater than the diameter of the micro-guidewire. Inserting the micro-guidewire through the centrally formed space of the catheter body portion (103), as described above, ensures that the center of the catheter and the center of the micro-guidewire are aligned in a straight line, significantly reducing the risk of catching on vessel walls or tissue during insertion, thus enabling faster placement.

The present invention has been described with reference to the above embodiments, which are merely illustrative. Those skilled in the art will understand that various modifications and equivalent embodiments are possible from this disclosure. Accordingly, the true technical scope of protection of the present invention shall be defined by the appended claims.

### DESCRIPTION OF REFERENCE NUMBERS IN THE DRAWINGS

| | |
|---|---|
| 100: a peripherally | |
| inserted central catheter | |
| 101: an end hole, | 102: a first side hole |
| 103: a catheter body portion, | 104: a catheter distal end portion |
| 105: a dilator, | 106: a peel-away sheath |
| 110: an introducer assembly, | 111: a stiffener |
| 202: a second side hole | |
| 203: a third side hole | |
| 204: an internal space | |
| 302: a fifth side hole | |
| 303: a sixth side hole | |
| 304: a seventh side hole | |
| 401: a first lumen | |
| 402: a second lumen | |
| 403: a third lumen | |
| 404: internal partition | |
| 500: blocking partition | |

1. A catheter kit for peripheral vascular insertion comprising:
a catheter body portion having a cylindrical structure configured to be inserted through a human blood vessel and having a lumen extending entirely therethrough;
a perfusion side hole formed on one side of the catheter body portion and configured to discharge an injected fluid, which is introduced from the catheter body portion into the human body;
a catheter distal end portion extending from the catheter body portion and having a tapering structure that gradually narrows toward its terminal end;
a peripherally inserted central catheter comprising an end hole for insertion of a micro-guided wire which is formed at the center of the catheter distal end portion; and
a dilator configured to dilate the vessel lumen by being inserted into the blood vessel.

2. The kit according to claim 1, wherein the peel-away sheath is not included.

3. The kit according to claim 1, wherein the dilator has a diameter equal to or reduced within 10% of the diameter of the peripherally inserted central catheter.

4. The kit according to claim 1, wherein the diameter of the end hole is increased within 10% of the diameter of the inserted micro-guidewire.

5. The kit according to claim 1, wherein the interior of the catheter main body is separated by one or more internal partitions, and is manufactured as a single lumen, double lumen, triple lumen, or multi-lumen according to the shape of the internal partitions.

6. The kit according to claim 5, wherein the double lumen comprises:
a main lumen extending entirely through the catheter main body; and
a sub-lumen separated from the main lumen by the internal partition and comprising a blocking partition located in front of the side hole.

7. The kit according to claim 5, wherein the triple lumen comprises:
a single main lumen extending entirely through the catheter main body; and
a plurality of sub-lumens which are separated from the main lumen by two or more internal partitions and comprise a plurality of blocking partitions in front of the side holes.

8. The kit according to claim 6, wherein the catheter distal end portion and the lower space in front of the blocking partition are filled with a material identical or similar to the catheter main body so that the step between the end hole and the internal partition is minimized, or the lower part of the catheter tube in front of the catheter distal end portion and the blocking partition is integrally structured in a thick form.

9. The kit according to claim 8, wherein the material is selected from the group consisting of silicone, polyurethane (PU), polytetrafluoroethylene (PTFE, Teflon), polyvinyl chloride (PVC), polystyrene, nylon, polyethylene terephthalate (PET), polyacrylate, polypropylene (PP), polyethylene (PE), polycarbonate (PC), polyether ether ketone (PEEK), and polysulfone (PS).

10. The kit according to claim 5, wherein the double lumen comprises:
a main lumen extending entirely through the catheter main body; and
a shorter sub-lumen which is separated from the main lumen by the internal partition and comprises a blocking partition at the end.

11. The kit according to claim 5, wherein the double lumen is configured such that a space is formed in the center of the internal partition when external force is applied vertically or laterally, allowing insertion of a micro-guidewire.

12. The kit according to claim 1, wherein the thickness of the sidewall forming the catheter main body and the catheter distal end portion is uniform.

13. The kit according to claim 1, wherein the injected substance into the catheter main body is blood, fluid, or drug.

14. A catheter kit for peripheral vascular insertion comprising:
a catheter body portion having a cylindrical structure configured to be inserted through a human blood vessel and having a lumen extending entirely therethrough;
a peripherally inserted central catheter comprising a perfusion side hole formed on a side of the catheter body portion for discharging an injected fluid, which has been introduced from inside the catheter body portion into the human body; and
a stiffener comprising a body insertable into the interior of the catheter body portion, a tapered distal end portion formed at the terminal end of the body, and an end hole formed at the center of the body for insertion of a micro-guide wire.

15. The kit according to claim 14, wherein the diameter of the end hole is increased within 10% of the diameter of the inserted micro-guidewire.

16. The kit according to claim 14, wherein the outer diameter of the stiffener is equal to or reduced within 10% of the inner diameter of the catheter main body.

17. The kit according to claim 7, wherein the catheter distal end portion and the lower space in front of the blocking partition are filled with a material identical or similar to the catheter main body so that the step between the end hole and the internal partition is minimized, or the lower part of the catheter tube in front of the catheter distal end portion and the blocking partition is integrally structured in a thick form.

18. The kit according to claim 17, wherein the material is selected from the group consisting of silicone, polyurethane (PU), polytetrafluoroethylene (PTFE, Teflon), polyvinyl chloride (PVC), polystyrene, nylon, polyethylene terephthalate (PET), polyacrylate, polypropylene (PP), polyethylene (PE), polycarbonate (PC), polyether ether ketone (PEEK), and polysulfone (PS).
